(19) United States
(12) Patent Application Publication
(10) Pub. No.: US 2013/0046145 A1
(43) Pub. Date: Feb. 21, 2013

(54) ENDOSCOPE SYSTEM

(71) Applicants: Hiroshi SHIBUYA, Ashigarakami-gun (JP); Kenji YAMAKAWA, Ashigarakami-gun (JP); Teppei EJIRI, Saitama-shi (JP)

(72) Inventors: Hiroshi SHIBUYA, Ashigarakami-gun (JP); Kenji YAMAKAWA, Ashigarakami-gun (JP); Teppei EJIRI, Saitama-shi (JP)

(73) Assignee: Fujifilm Corporation, Tokyo (JP)

(21) Appl. No.: 13/545,912

(22) Filed: Jul. 10, 2012

(30) Foreign Application Priority Data
Aug. 17, 2011 (JP) JP2011-178495

(51) Int. Cl. A61B 1/015

(52) U.S. Cl. 600/157

(57) ABSTRACT

An endoscope system includes a nozzle provided at a tip of a tube forming member, communicating with a fluid tube, and injecting a fluid supplied from the fluid tube toward a window part of an endoscope insertion part, a first gas supply tube provided between a gas supply source and the fluid tube and guiding a gas to the fluid tube, a second gas supply tube having a limiting device limiting a pressure or a flow rate of the gas supplied from the gas supply source, the second gas supply tube guiding the gas with the pressure or the flow rate limited by the limiting device to the fluid tube, and a gas switching device provided only to the first gas supply tube between the first and second gas supply tubes and selectively switching between supplying and stopping of the gas to the fluid tube.

### BACKGROUND OF THE INVENTION

1. Field of the Invention

The present invention relates to an endoscope system and, in particular, to an endoscope system including a mechanism for forming a gas curtain on the surface of an observation window provided at the tip of an endoscope insertion part for preventing a taint and fogging of the observation window.

2. Description of the Related Art

In medical fields, medical diagnoses using endoscopes are widely used. Since an insertion part of an endoscope is used under an environment where it is easily tainted with blood, body fluid, and others, it is required to always keep an observation window provided at the tip of the endoscope's insertion part clean to ensure a good field of view.

In a rigid endoscope described in Japanese Patent Application Laid-Open No. 5-199979, a nozzle for issuing a jet of a fluid such as a cleaning solution or gas is provided at the tip of a sheath with which an insertion part of the rigid endoscope. In a space formed of an inner wall of this sheath, a liquid-supply tube and an air-supply tube are provided as fluid tubes formed along an axial direction. These fluid tubes merge near the tip of the sheath to communicate with the nozzle provided at the tip of the sheath. Also, the pressure of a gas with which a gas cylinder is filled as a gas supply source is reduced to a predetermined pressure to be supplied to a container having accommodated therein a cleaning solution such as physiological saline. To this container, one ends of a liquid-supply tube and an air-supply tube are connected, and the other end of the respective tubes are connected to a liquid-supply inlet body and an air-supply inlet body provided at the sheath to communicate with the water tube and the air tube of the sheath, respectively. When blood or body fluid is attached onto an observation window at the tip of the insertion part of the endoscope during observation or treatment of an organ in an abdominal cavity with the rigid endoscope, the gas supplied from the gas cylinder is used to cause the cleaning solution or the gas to be guided from the container via the fluid tube path of the sheath to the nozzle and then be supplied from the nozzle. In this manner, the observation window of the endoscope's insertion part is cleaned and dried, thereby allowing the field of view of the observation window to be kept good.

Meanwhile, the patent gazette mentioned above describes that a jet of gas is issued from the nozzle provided at the tip of the sheath to form a gas curtain (air curtain) on the surface of the observation window of the endoscope insertion part. As such, by forming a gas curtain, a taint and fogging of the observation window can be prevented before occurring, thereby producing a desirable effect of allowing a decrease of the frequency of operations for cleaning and drying the observation window and a reduction of an operation load on a person who is conducting an operation and others.

Here, when the rigid endoscope device of the patent gazette described above is used to form a gas curtain on the surface of the observation window of the endoscope's insertion part, it can be thought that a gas for pneumoperitoneum with its pressure reduced to an air pressure or a cleaning pressure is supplied to the air-supply tube and a jet of the gas is issued from the nozzle.

However, when a gas for pneumoperitoneum with its pressure reduced to an abdominal air pressure is used, since the gas for pneumoperitoneum is a gas for inflating an abdominal cavity to a predetermined pressure, if the pressure in the abdominal cavity becomes higher than the predetermined pressure, an air supply of the gas for pneumoperitoneum to the abdominal cavity has to be stopped in order to avoid a high-pressured state in the abdominal cavity. For this reason, there is a problem such that the gas for pneumoperitoneum for forming a gas curtain on the surface of the observation window cannot be always supplied to the abdominal cavity during an observation or treatment of an organ or the like in the abdominal cavity with the rigid endoscope, and thus a gas curtain cannot be always formed on the surface of the observation window. As a result, an effect of suppressing a taint and fogging of the observation window cannot be sufficiently obtained, and the observation window is required to be cleaned and dried afterward.

On the other hand, since the pressure of the gas for pneumoperitoneum reduced to the clearing pressure is higher than the pressure of the gas for pneumoperitoneum reduced to the abdominal air pressure, the problem described above is significant, and the gas for pneumoperitoneum reduced to the clearing pressure is not preferable as a gas to be used for forming a gas curtain. Moreover, always issuing a jet of such a high-pressure gas from the nozzle wastefully consumes the gas more than required, and is therefore uneconomical.

Furthermore, the rigid endoscope device of the patent gazette described above is configured in a manner such that a first gas tube corresponding to the cleaning pressure and a second gas tube corresponding to the abdominal air pressure merge to be connected to the container via a connection tube. Thus, a switching mechanism (such as a valve mechanism) for switching between these tubes and a control device are required, thereby possibly inviting complication in operation and an increase in cost due to an increase in the size of the system.

The present invention was made in view of these circumstances, and has an object of providing an endoscope system capable of always forming a gas curtain on the surface of a window part of an endoscope insertion part without making operations complex and with a simple structure to prevent a taint and fogging of the window part.

### SUMMARY OF THE INVENTION

To achieve the object described above, an endoscope system according to the present invention includes an endoscope insertion part having a window part at an end face, a fluid tube provided to a tube forming member formed of the endoscope insertion part or a sheath sheathing the endoscope insertion part, a nozzle provided at a tip of the tube forming member, communicating with the fluid tube, and injecting a fluid supplied from the fluid tube toward the window part of the endoscope insertion part, a first gas supply tube provided between a gas supply source and the fluid tube and guiding a gas supplied from the gas supply source to the fluid tube, a second gas supply tube having limiting device limiting a pressure or a flow rate of the gas supplied from the gas supply source, the second gas supply tube guiding the gas with the pressure or the flow rate limited by the limiting means to the fluid tube; and a gas switching device provided only to the first gas supply tube between the first and second gas supply tubes and selectively switching between supplying and stopping of the gas to the fluid tube.

According to the present invention, together with the first gas supply tube guiding the gas supplied from the gas supply source to the fluid tube, the second gas supply tube guiding the gas with its flow rate limited to be low or with low pressure by the limiting device (a gas-curtain gas) to the fluid tube is provided. Thus, a jet of the gas-curtain gas can be always issued from the nozzle without a complicated operation. With this, a gas curtain can always be formed on the surface of the window part of the endoscope insertion part, and fogging and a taint on the window part (for example, an observation window or an illumination window) of the endoscope insertion part can be prevented before occurring.

In the present invention, preferably, the first gas supply tube is a gas supply tube guiding a gas for cleaning or drying the window part of the endoscope insertion part to the fluid tube, and the second gas supply tube is a gas supply tube guiding a gas to the fluid tube, the gas for forming a gas curtain on a surface of the window part of the endoscope insertion part and having a pressure or a flow rate lower than the pressure or the flow rate of the gas guided by the first gas supply tube.

Also, as a preferable embodiment of the present invention, the endoscope system further includes a cleaning-fluid supply tube provided between the gas supply source and the fluid tube and guiding a cleaning solution accommodated in an air-tight container to the fluid tube by using the pressure of the gas supplied from the gas supply source; and cleaning-fluid switching device provided to the cleaning-fluid supply tube and selectively switching between supplying and stopping of the cleaning solution to the fluid tube. According to this embodiment, by selectively injecting the cleaning solution from the nozzle, a taint and a droplet attached onto the window part of the endoscope insertion part can be washed out.

Furthermore, as another preferable embodiment of the present invention, the gas switching device and the cleaning-fluid switching device are configured to have timings of opening and closing the gas supply tube and the cleaning-fluid supply tube, respectively, varying according to an amount of operation of an operating member. According to this embodiment, the gas or the cleaning solution can be injected from the nozzle according to the amount of operation of the operating member. With this, the window part of the endoscope insertion part can be cleaned and dried with a simple operation.

Still further, in the present invention, preferably, the flow rate of the gas limited by the limiting device and guided by the second gas supply tube is equal to 1/100 or more and equal to 1/50 or less of the gas guided by the first gas supply tube. By using the gas with the flow rate set as described above, a gas curtain can be stably formed on the window part of the endoscope insertion part.

According to the present invention, together with the first gas supply tube guiding the gas supplied from the gas supply source to the fluid tube, the second gas supply tube guiding the gas with its flow rate limited to be low or with low pressure by the limiting device (a gas-curtain gas) to the fluid tube is provided. Thus, a jet of the gas-curtain gas can be always issued from the nozzle without a complicated operation. With this, a gas curtain can always be formed on the surface of the window part of the endoscope insertion part, and fogging and a taint on the window part (for example, an observation window or an illumination window) of the endoscope insertion part can be prevented before occurring.

### BRIEF DESCRIPTION OF THE DRAWINGS

FIG. 1 is a perspective view showing an entire structure of an endoscope system according an embodiment of the present invention;

FIG. 2 is a perspective view of the endoscope system shown in FIG. 1 as being viewed from below;

FIG. 3 is an exploded perspective view of the endoscope system shown in FIG. 1;

FIG. 4 is a schematic view showing the structure of a fluid supply system in the endoscope system of the present embodiment;

FIGS. 5A, 5B, and 5C are schematic sectional views showing an open/closed state of a tube at time of operation of an operating lever;

FIGS. 6A and 6B are schematic sectional views showing an open/closed state of the tube at the time of operation of the operating lever;

FIG. 7 is a sectional view showing the state in which a closing-preventive member is inserted in an air-supply tube;

FIG. 8 is a sectional view showing the structure in which a projection is provided in the air-supply tube;

FIG. 9 is a sectional view showing the structure in which a recessed part is provided in the air-supply tube;

FIG. 10 is a sectional view showing the state in which an air-supply tube is pressed into a V-shape groove part; and

FIGS. 11A and 11B are sectional views showing the structure in which a bypass tube is connected to the air-supply tube.

### DETAILED DESCRIPTION OF THE PREFERRED EMBODIMENTS

Preferred embodiments of an endoscope system according to the present invention are described in detail below according to the attached drawings.

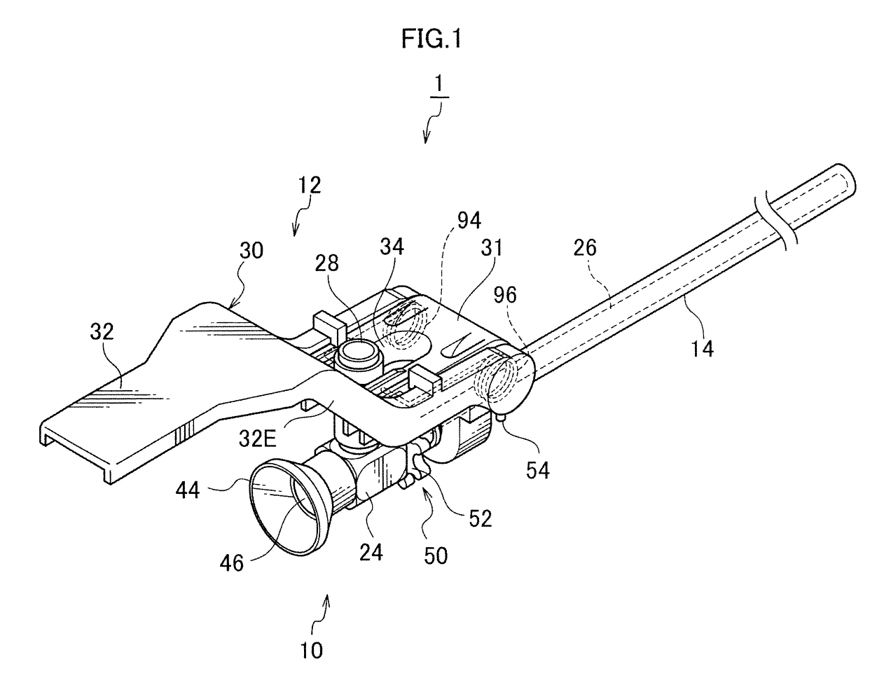

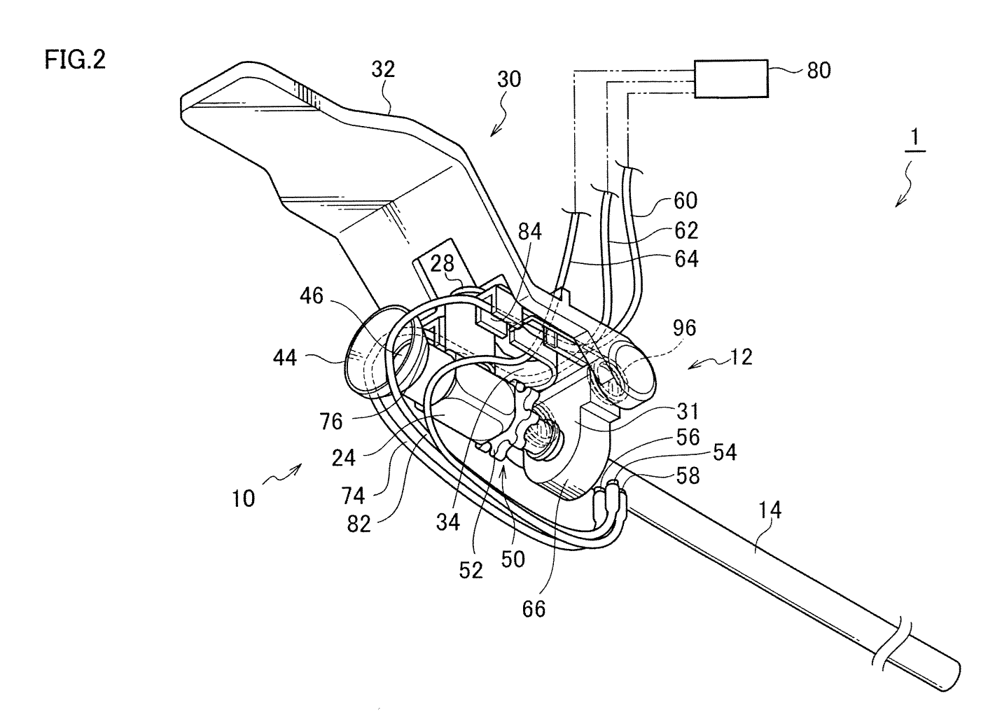

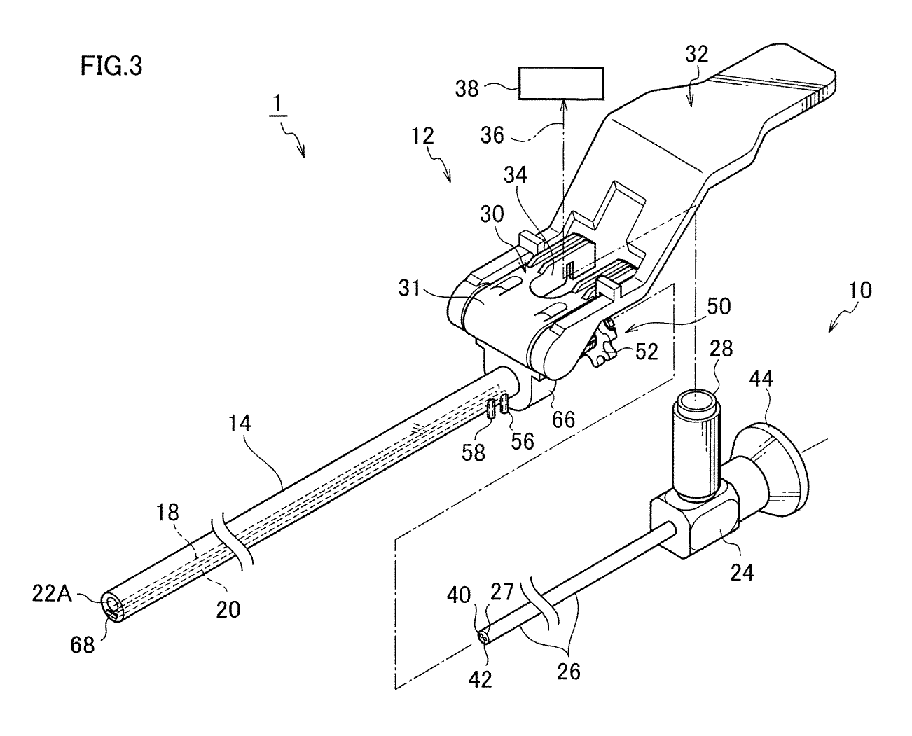

FIG. 1 is a perspective view of an entire structure of an endoscope system according an embodiment of the present invention. FIG. 2 is a perspective view of the endoscope system shown in FIG. 1 as being viewed from below. FIG. 3 is an exploded perspective view of the endoscope system shown in FIG. 1, showing the state in which a sheath and a rigid endoscope are separated from each other. Note that for simplification of the drawings, part of components is not shown in each drawing.

As shown in FIG. 1 to FIG. 3, an endoscope system 1 according to the present embodiment is mainly configured of a rigid endoscope 10, a sheath 12 sheathing the tip portion of the rigid endoscope 10, and a fluid supply device 80 (refer to FIG. 2) for supplying a cleaning solution or an air-supply gas to a fluid tube provided inside the sheath 12.

The rigid endoscope 10 is configured of an elongated, rigid insertion part (hereinafter referred to as an “endoscope insertion part”) 26 and a body part (hereinafter referred to as an “endoscope body part”) 24 coupled to a base end of the endoscope insertion part (refer to FIG. 3).

To the endoscope body part 24, a connector 28 for connecting a light source device 38 is provided so as to project. This connector 28 is arranged so as to project upward from a U-shaped notch 34 of a body part 31 of a sheath operating part 30 provided at a base end of the sheath 12 at the time of using the rigid endoscope 10, that is, as shown in FIG. 1 and FIG. 2, a the time when the sheath 12 is inserted in the rigid endoscope 10. In this state, one end of a light guide cable 36 is connected to the connector 28, and the other end of the light guide cable 36 is connected to the light source device 38 provided outside.

An illumination window 40 is provided on an end face 27 of the endoscope insertion part 26. In the endoscope insertion part 26, an outgoing light end of a light guide (not shown) is arranged behind the illumination window 40. This light guide is inserted in the endoscope insertion part 26, and an incoming light end of the light guide is arranged in the connector 28. With this, illumination light from the light source device 38 is guided via the light guide cable 36 and the light guide to the illumination window 40, and is applied from the illumination window 40 to a region to be observed in an abdominal cavity.

Also, an observation window 42 is provided on the end face 27 of the endoscope insertion part 26. In the endoscope insertion part 26, an objective lens (not shown) and an image guide (not shown) are arranged behind the observation window 42. An optical image of the region to be observed entering from the observation window 42 is formed by the objective lens on an image plane of the image guide. Then, the optical image is guided via the image guide to the endoscope body part 24. An ocular part 44 is coupled to a base end of the endoscope body part 24. The optical image guided by the endoscope body part 24 is observed by a person who is conducting an operation or treatment via an ocular lens 46 (refer to FIG. 1 and FIG. 2) of the ocular part 44. Note that while the rigid endoscope 10 for observing the optical image of the region to be observed from the ocular part 44 via the image guide has been exemplarily described in the present embodiment, an electronic endoscope for forming an optical image on a light-receiving plane of a solid-state imaging device and outputting the optical image as electronic image data may be used.

The sheath 12 is configured of an insertion part (hereinafter referred to as a “sheath insertion part”) 14 formed of an elongated hard pipe in a simple cylindrical shape and the sheath operating part 30 coupled to a base end of the sheath insertion part 14.

In the sheath insertion part 14, a lumen 16 for letting the endoscope insertion part 26 be inserted to pass through along an axial direction (hereinafter referred to as a “sheath axial direction”) is formed. This lumen 16 has a diameter slightly larger than the outer diameter of the endoscope insertion part 26 and to such a degree that the endoscope insertion part 26 hardly rattles in the sheath insertion part 14 and the endoscope insertion part 26 can be smoothly inserted in the sheath insertion part 14. Note that the sheath insertion part 14 is preferably made of a resin such as polyethylene, polypropylene, or urethane.

The sheath insertion part 14 is formed so as to match the length of the endoscope insertion part 26. Specifically, it is configured that a tip of the sheath insertion part 14 approximately match an end face of the endoscope insertion part 26 when the endoscope insertion part 26 is inserted in the sheath insertion part 14 to a set position.

A collet chuck 50 is provided at the base end of the sheath insertion part 14 as a fixing mechanism for fixing the endoscope insertion part 26. With the sheath insertion part 14 being inserted to pass through the endoscope insertion part 26, a nut part 52 configuring the collet chuck 50 is rotated in a fastening direction, thereby fixing the sheath insertion part 14 and the endoscope insertion part 26 onto each other. Note that the structure of the collet chuck 50 is well known and therefore its specific structure is not described herein. A nozzle 68 is provided at a tip of the sheath insertion part 14 as means injecting a fluid such as a cleaning solution or gas for removing a fouling substance (for example, blood or body fluid) attached onto the observation window 42 of the endoscope insertion part 26. This nozzle 68 is bent in a direction approximately at right angles with respect to the sheath axial direction or approximately parallel to an end face of a hard mirror to be coupled, and opens toward the observation window 42 of the endoscope insertion part 26 inserted to pass through the sheath insertion part 14. In a space formed by an inner wall of the sheath insertion part 14, a liquid-supply tube 18 and an air-supply tube 20 are provided as fluid tubes for guiding fluids such as a cleaning solution and gas to the nozzle 68 (refer to FIG. 3). These fluid tubes are formed along the sheath axial direction, and merge near the tip of the sheath insertion part 14 to communicate with the nozzle 68 provided at the tip of the sheath insertion part 14. With this, a fluid supplied from a fluid supply device 80, which will be described further below, is guided via a predetermined fluid tube (the liquid-supply tube 18 or the air-supply tube 20) to the nozzle 68, and is injected from the nozzle 68 toward the observation window 42. Note that the liquid-supply tube 18 and the air-supply tube 20 may be configured communicate with the nozzle 68 separately without merging.

At the base end of the sheath insertion part 14, a liquid-supply port 54 and air-supply ports 56 and 58 are provided as supply inlets for introducing fluids supplied from the fluid supply device 80 as an external device to the fluid tubes of the sheath insertion part 14.

The liquid-supply port 54 communicates with the liquid-supply tube 18, and has one end of a liquid-supply tube 60 connected thereto. The other end of the liquid-supply tube 60 is connected to a liquid-supply connector 80d of the fluid supply device 80. The cleaning fluid supplied from the liquid-supply connector 80d of the fluid supply device 80 is guided via the liquid-supply tube 60, the liquid-supply port 54, and then the liquid-supply tube 18 to the nozzle 68.

The air-supply port 56 communicates with the air-supply tube 20, and has one end of an air-supply tube 62 connected thereto. The other end of the air-supply tube 62 is connected to an air-supply connector 80c of the fluid supply device 80. A gas supplied from the air-supply connector 80c of the fluid supply device 80 is a high-pressure gas (a blow gas) for blowing a taint or droplet attached onto the observation window 42 of the endoscope insertion part 26. The gas supplied from the air-supply connector 30c of the fluid supply device 80 is guided via the air-supply tube 62, the air-supply port 56, and then the air-supply tube 20 to the nozzle 68.

The air-supply port 58 communicates with the air-supply tube 20, and has one end of an air-supply tube 64 connected thereto. The other end of the air-supply tube 64 is connected to an air-supply connector 80b of the fluid supply device 80. A gas supplied from the air-supply connector 80b of the fluid supply device 80 is a gas (a gas-curtain gas) for forming a gas curtain on the surface of the observation window 42 to remove fogging and a taint from the observation window 42 of the endoscope insertion part 26, and is set at a pressure lower than that of the blow gas. The gas supplied from the air-supply connector 80b of the fluid supply device 80 is guided via the air-supply tube 64, the air-supply port 58, and then the air-supply tube 20 to the nozzle 68.

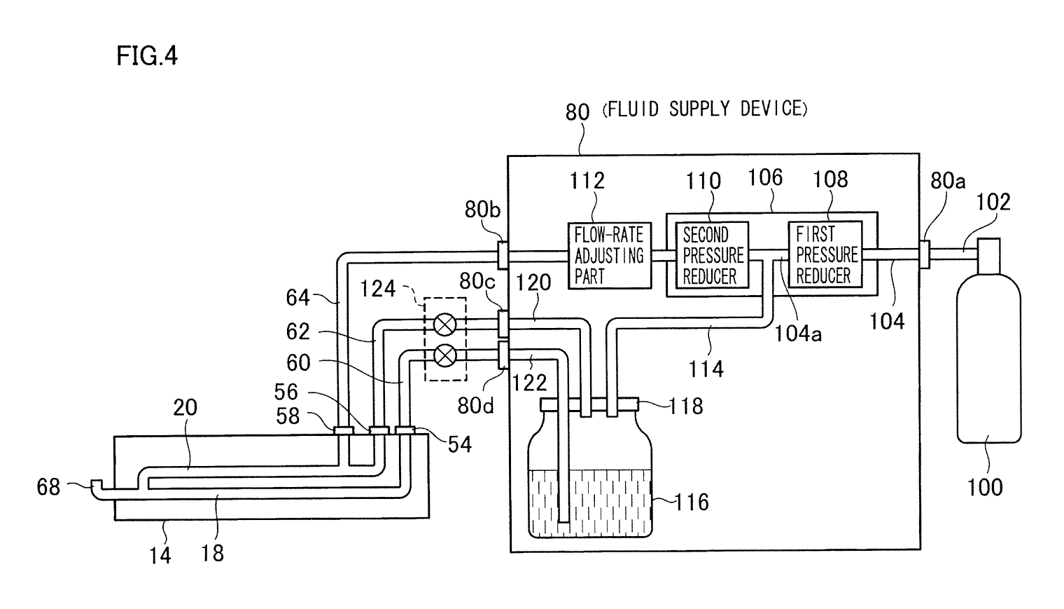

Here, the structure of the fluid supply device 80 is described. FIG. 4 is a schematic view showing the structure of a fluid supply system in the endoscope system of the present embodiment.

As shown in FIG. 4, the fluid supply device 80 is provided with a plurality of connectors 80a to 80d. As described above, the tubes 60, 62, and 64 are connected to the connectors 80d, 80c, and 80b, respectively.

To the high-pressure connector 80a of the fluid supply device 80, one end of a high-pressure-gas tube 102 extending from a gas cylinder 100 is connected. The gas cylinder 100 as a gas supply source is filled with carbon dioxide gas (CO₂ gas), and the carbon dioxide gas is supplied from the gas cylinder 100 via the high-pressure-gas tube 102 to the high-pressure connector 80a of the fluid supply device 80.

Inside the fluid supply device 80, a gas tube 104 coupled to the high-pressure connector 80a is provided. This his gas tube 104 is provided with a pressure reducing unit 106 reducing the pressure of the gas (in this example, carbon dioxide gas) supplied to the high-pressure connector 80a to a predetermined pressure and a flow-rate adjusting part 112 adjusting the flow rate of the gas flowing through the gas tube 104.

In the pressure reducing unit 106, a first pressure reducer 108 and a second pressure reducer 110 are connected in series in this order from an upstream side (a high-pressure connector 80a side) of the gas tube 104. The first pressure reducer 108 reduces the pressure of the gas supplied to the high-pressure connector 80a to a primary pressure (a blow pressure) suitable for blowing a taint and a droplet attached onto the surface of the observation window 42. The second pressure reducer 110 reduces the pressure of the gas reduced by the first pressure reducer 108 to a secondary pressure (a gas-curtain pressure) suitable for forming a gas curtain. Note that the second pressure reducer 110 can be omitted when the flow-rate adjusting part 112, which will be described further below, is provided.

The flow-rate adjusting part 112 adjusts the flow rate of the gas flowing through the gas tube 104 to a predetermined flow rate by changing the cross-sectional area (a tube area) of the gas tube 104. This flow-rate adjusting part 112 is configured of, for example, a variable diaphragm or a flow-rate control valve capable of varying the cross-sectional area of the tube. Note that the flow-rate adjusting part 112 can be omitted when the second pressure reducer 110 is provided.

To a gas tube 104a connecting the first pressure reducer 108 and the second pressure reducer 110, one end of a branch tube 114 is connected, and the other end of the branch tube 114 is connected to a container 116. The container 116 has a cleaning fluid (for example, physiological saline) accommodated therein, and is hermetically sealed with a lid body 118. The other end of the branch tube 114 is arranged so as to penetrate through the lid body 118, and opens above a fluid level of the cleaning fluid.

One end of an air-supply tube 120 is arranged to penetrate through the lid body 118, and opens above the fluid level of the cleaning fluid in the container 116. The other end of the air-supply tube 120 is connected to the air-supply connector 80c of the fluid supply device 80.

One end of a liquid-supply tube 122 is arranged to penetrate through the lid body 118, and opens in the cleaning fluid near the bottom of the container 116. The other end of the liquid-supply tube 122 is connected to the liquid-supply connector 80d of the fluid supply device 80.

In the fluid supply device 80 as described above, the gas supplied from the gas cylinder 100 via the high-pressure-gas tube 102 to the high-pressure connector 80a of the fluid supply device 80 sequentially passes the first pressure reducer 108, the second pressure reducer 110, and the flow-rate adjusting part 112 to be adjusted so as to have a pressure or a flow rate suitable for forming a gas curtain, and is then outputted from the air-supply connector 80b. Also, the gas with its pressure reduced by the first pressure reducer 108 to the blow pressure suitable for blowing a taint and a droplet attached onto the surface of the observation window 42 is outputted from the air-supply connector 80c via the container 116, and the cleaning fluid in the container 116 is outputted from the liquid-supply connector 80d by using the pressure of the gas reduced to the blow pressure. Then, the gas and the cleaning fluid sent from the connectors 80d, 80c, and 80b are supplied via the tubes 60, 62, and 64, respectively, each to a relevant fluid tube (the liquid-supply tube 18 or the air-supply tube 20) of the sheath insertion part 14.

Meanwhile, an operating mechanism (an ON/OFF mechanism) 124 for supplying/stopping the cleaning fluid and the blow gas (the gas with its pressure reduced to the blow pressure) is provided between the fluid supply device 80 and the sheath insertion part 14. This operating mechanism 124 is incorporated as a component of the sheath operating part 30. According to the operation of the sheath operating part 30, the tube configured of the liquid-supply tube 60 or the air-supply tube 62 is opened/closed to supply/stop the cleaning fluid or gas flowing through each relevant tube. With this, injection of the cleaning fluid or the gas from the nozzle 68 at the tip of the sheath insertion part 14 is selectively performed.

Note that no mechanism is provided for supplying/stopping the gas-curtain gas (the gas with its pressure or flow rate adjusted to be relatively low for forming a gas curtain) is not provided between the fluid supply device 80 and the sheath insertion part 14. From the fluid supply device 80 to the sheath insertion part 14, the gas-curtain gas is always supplied, and a gas curtain is always formed on the surface of the observation window 42 with the gas injected from the nozzle 68 at the tip of the sheath insertion part 14.

Here, the structure of the sheath operating part 30 is described in detail.

As shown in FIG. 1 to FIG. 3, the sheath operating part 30 is configured of the body part 31 coupled and fixed to the sheath insertion part 14 via a support part 70, an operating lever 32 rotatably provided to the body part 31, and helical torsion coil springs 94 and 96 as pressing means for closing the liquid-supply tube 60 and the air-supply tube 62.

The body part 31 is provided with the U-shaped notch 34 for inserting the connector 28 of the endoscope body part 24 to pass therethrough as described above, and is also provided with grooves 76 and 84 on a lower surface side for disposing tubes, the grooves interposing the U-shaped notch 34 therebetween (refer to FIG. 2). Part of the air-supply tube 62 is arranged in the first groove 76, and part of the liquid-supply tube 60 is arranged in the second groove 84. At the time of a non-operating state of the operating lever 32, the tubes 62 and 60 arranged in these grooves 76 and 84 are in a closed state by the pressing force of the helical torsion coil springs 94 ad 96, respectively.

The operating lever 32 is an operating member for releasing the closing of each of the tubes 62 and 60 closed by the helical torsion coil springs 94 and 96, respectively, and is operable (pressable) in two steps with a shaft part 98 provided to the body part 31. The operating lever 32 is configured so that, in response to the rotating operation (pressing operation) of the operating lever 32, part of the operating lever 32 abuts on the helical torsion coil springs 94 and 96, thereby allowing the closing of each of the tubes 62 and 60 to be released. With this, it is configured that switching is made between a closed state produced by pressing (crushing) any of the air-supply tube 62 and the liquid-supply tube 60 arranged in the grooves 76 and 84, respectively, to close the tube of the relevant tube and an open state produced by releasing the pressing (crushing) of a relevant one of the tubes to release the tube of the tube.

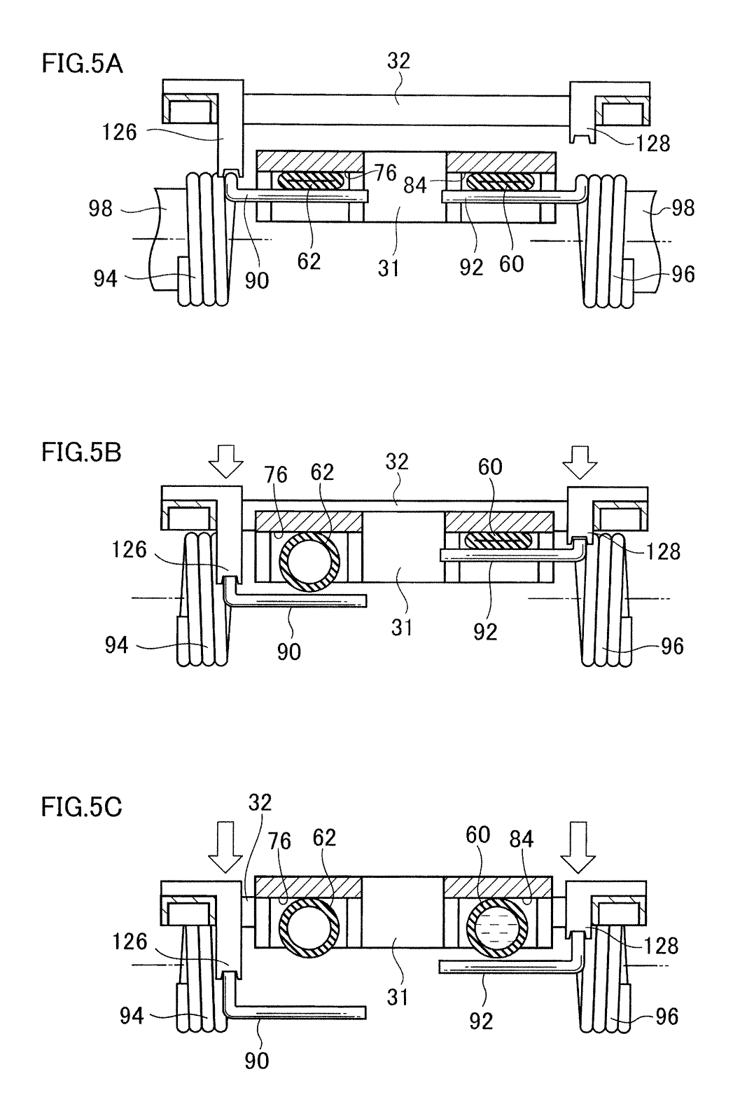

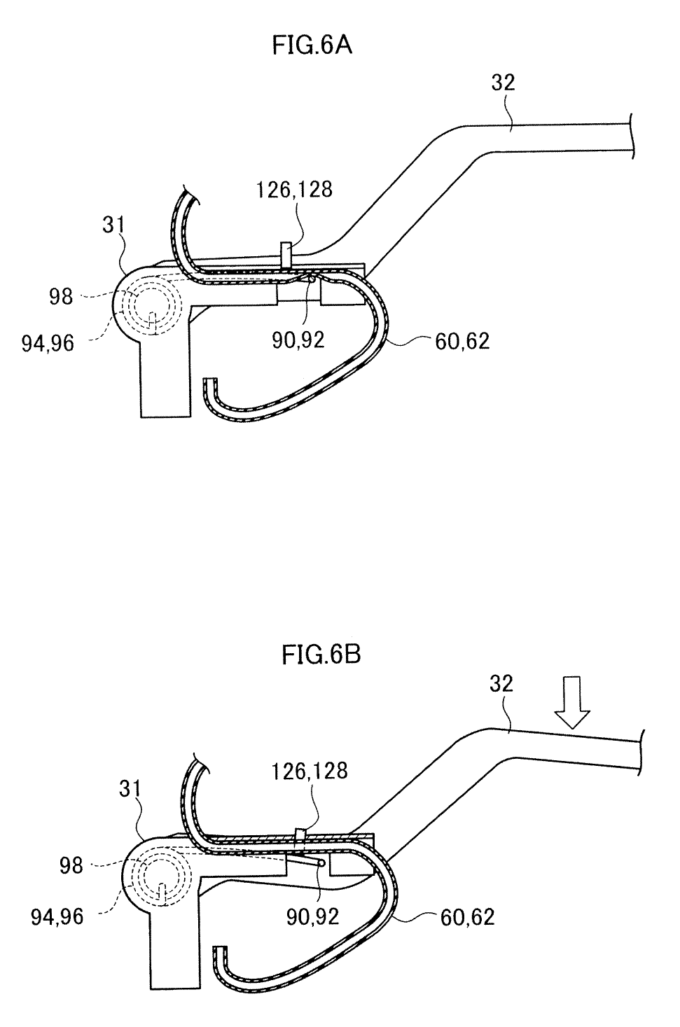

FIGS. 5A, 5B, 5C, 6A, and 6B are schematic sectional views showing an open/closed state of the tube at the time of operation of the operating lever. FIGS. 5A, 5B, and 5C show the state of the operating lever when viewed from a back surface side (a side opposite to the tip of the sheath insertion part 14), and FIGS. 6A and 6B show the state of the operating lever when viewed from a side surface side.

First, in the state at the time of non-operation of the operating lever 32 as shown in FIG. 5A and FIG. 6A, abutting members (a pressing members) 126 and 128 projected to a lower surface side (a back surface side) of the operating lever 32 are configured not to abut on the helical torsion coil springs 94 and 96, respectively. Here, stoppers 90 and 92 configured by folding one ends of the helical torsion coil springs 94 and 96 at right angles are pressed with pressing forces by the helical torsion coil springs 94 and 96 in a direction in which the air-supply tube 62 and the liquid-supply tube 60 are pressed, respectively (in an upper direction in FIG. 5A). The tubes 62 and 60 are in a closed state with the stoppers 90 and 92, respectively. That is, the tubes of the air-supply tube 62 and the liquid-supply tube 60 are both in a closed state.

Also, in the state in which the operating lever 32 is pressed down one step further as shown in FIG. 5B and FIG. 6B, the first abutting member 126 between the first and second abutting members 126 and 128 of the operating lever 32 abuts on the helical torsion coil spring 94, and the stopper 90 is pressed down against the pressing force of the helical torsion coil spring 94. With this, the tube of the air-supply tube 62 is released to become in an open state, and the blow gas outputted from the air-supply connector 80c of the fluid supply device 80 is supplied via the air-supply port 56 and the air-supply tube 20 to the nozzle 68. On the other hand, the second abutting member 128 is configured to have a length shorter than that of the first abutting member 126. In the state in which the operating lever 32 is pressed down one step further, the second abutting member 128 does not abut on the helical torsion coil spring 94 and, as with the non-operating state shown in FIG. 5A, the liquid-supply tube 60 is in a closed state as being pressed by the stopper 92 to close the tube. Thus, in the state in which the operating lever 32 is pressed down one step further, only the blow gas is injected from the nozzle 68 at the tip of the sheath insertion part 14.

Furthermore, in the state in which the operating lever 32 is pressed down two steps further as shown in FIG. 5C, the state in which the abutting member 126 abuts on the helical torsion coil spring 94 is kept, and the stopper 90 is further pressed down against the pressing force of the helical torsion coil spring 94 and the pressing member 128 abuts on the helical torsion coil spring 96 to press the stopper 92 down against the helical torsion coil spring 96. With this, the tube of the liquid-supply tube 60 and the tube of the air-supply tube 62 are both opened to become in an open state. With this, a mixed fluid of the cleaning fluid and the blow gas is injected from the nozzle 68 at the tip of the sheath insertion part 14.

Next, the operation of the endoscope system 1 of the present embodiment is described.

When an organ or the like in an abdominal cavity is observed or treated by the rigid endoscope 10, the gas adjusted by the fluid supply device 80 to have a relatively low pressure or low flow rate for forming a gas curtain is introduced via the air-supply tube 64, the air-supply port 58, and the air-supply tube 20 to the nozzle 68, and is injected from the nozzle 68. Then, with the gas injected from the nozzle 68, a gas film is always formed on the surface of the observation window 42 and flows along the surface. With this, smoke, water vapor, and others toward the observation window 42 do not reach the surface of the observation window 42, and are conveyed by the gas curtain to flow away sideward from the observation window 42. Also, oil, blood, and others fly, and these may be attached onto the observation window 42. However, these are also blocked by the gas curtain, and it is therefore possible to prevent oil, blood, and others from being attached onto the observation window 42. That is, with the gas curtain, fogging or a taint of the observation window 42 can be prevented before occurring. Also, since no operating mechanism for opening and closing the tube is provided to the air-supply tube 64, a gas curtain can be always formed on the surface of the observation window 42 at the tip of the endoscope insertion part 26 without performing a special operation.

Furthermore, when a fouling substance is attached onto the observation window 42 at the tip of the endoscope insertion part 26, a rotating operation (a pressing operation) is first performed on the operating lever 32 from a position in a non-operating state (FIG. 5A) to an operation position of a first step (FIG. 5B) to remove the fouling substance. With this, the gas (the gas blow) with its pressure reduced by the fluid supply device 80 to a relatively high pressure for blowing is injected from the nozzle 68, thereby causing a taint or a droplet of blood, body fluid, and others attached onto the observation window 42 to be blown away.

Subsequently, when a pressing operation is performed on the operating lever 32 from the operating position of the first step (FIG. 5B) to an operating position of the second step (FIG. 5C), the mixed fluid of the blow gas and the cleaning fluid is injected from the nozzle 68 to the observation window 42, cleaning a taint or droplet attached onto the observation window 42.

Next, when the operating lever 32 is returned from the operating position of the second step (FIG. 5C) to the operating position of the first step (FIG. 5B), the blow gas is injected from the nozzle 68 to blow a droplet and others attached onto the observation window 42 away and dry the observation window 42.

Then, when the operating lever 32 is returned from the operating position of the first step (FIG. 5B) to the position in the non-operating state (FIG. 5A), that is, when the operating lever 32 is returned to be in an initial state, injection of the blow gas from the nozzle 68 is stopped. Here, since the gas-curtain gas is always supplied from the fluid supply device 80 to the air-supply tube 20 of the sheath insertion part 14, there is no time lag from the time when injection of the blow gas from the nozzle 68 is stopped to the time when the gas-curtain gas is injected, and a gas curtain can be formed simultaneously with the end of cleaning and drying the observation window 42. Also, a special operation for forming a gas curtain is not required, the operation load can be significantly reduced, and a taint and fogging of the observation window 42 can be reliably prevented.

According to the endoscope system 1 of the present embodiment, together with the air-supply tube 62 for supplying the high-pressure blow gas to the air-supply tube 20 of the sheath insertion part 14, the air-supply tube 64 for supplying the low-pressure gas-curtain gas to the air-supply tube 20 of the sheath insertion part 14 is provided. Therefore, a jet of the gas-curtain gas can be always issued from the nozzle 68 without depending on the operation for switching between supplying and stopping of the blow gas. With this, a gas curtain can be always formed on the surface of the observation window 42 of the endoscope insertion part 26, and fogging and a taint of the observation window 42 of the endoscope insertion part 26 can be prevented before occurring. Also, since the blow gas is injected from the nozzle 68 according to the operation of the operating lever 32, a taint or droplet attached onto the observation window 42 of the endoscope insertion part 26 can be blown away.

Furthermore, the gas-curtain gas is injected simultaneously with the end of injection of the blow gas from the nozzle 68, there is no time lag from the time when cleaning and drying the observation window 42 ends to the time when a gas curtain is formed, a taint of the observation window 42 can be reliably prevented, and a good field of view can be always kept.

In the present embodiment, a ratio Q in flow rate between the gas-curtain gas and the blow gas is 1/100≦Q≦ 1/50. Specifically, the flow rate of the gas flowing through the gas tube 104 is adjusted by the flow-rate adjusting part 112 of the fluid supply device 80 so that the ratio Q in flow rate between these gases. With this, a gas curtain can be stably formed on the surface of the observation window 42 of the endoscope insertion part 26, and wasteful gas consumption can be prevented.

Still further, while the operating mechanism (the ON/OFF mechanism) 124 for supplying/stopping the cleaning fluid and the blow gas is configured of a mechanical mechanism formed of the sheath operating part 30 in the present embodiment, the present invention is not meant to be restricted to this, and may be configured of an electrical mechanism.

Still further, while the structure is shown in the present embodiment in which the nozzle 68 and the fluid tubes (the liquid-supply tube 18 and the air-supply tube 20) communicating therewith are provided to the sheath insertion part 14, the present invention is not meant to be restricted to this, and may be configured in a manner such that the nozzle 68 and the fluid tubes are provided to the endoscope insertion part 26.

Note that when the operating lever 32 is rotated between the position in FIG. 5A and the position in FIG. 5B to release the air-supply tube 62 by a slight amount, a subtle amount of gas is injected from the nozzle 68 to the observation window 42. With this, a gas curtain can also be formed on the surface of the observation window 42. In this case, the air-supply tube 64 for supplying the gas-curtain gas is not required.

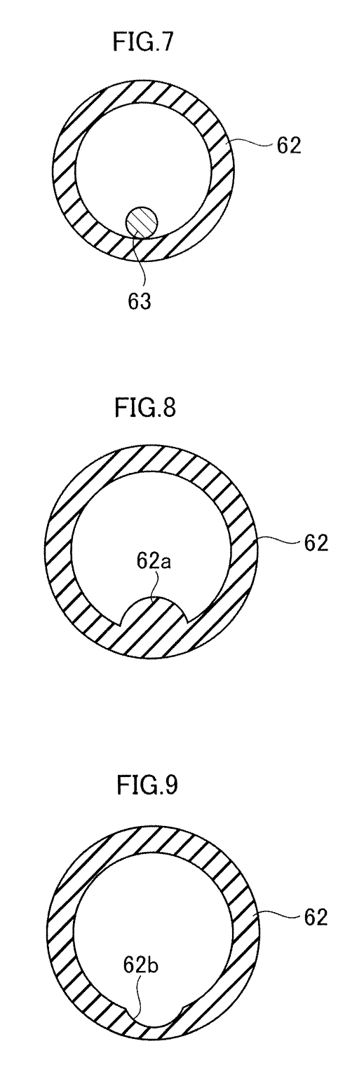

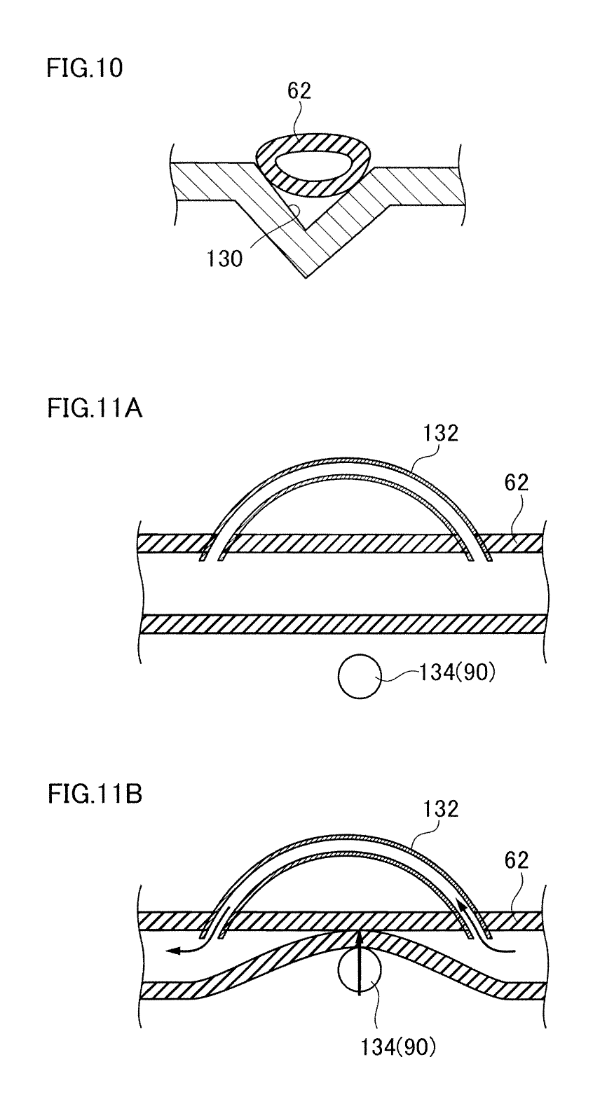

When a gas-curtain gas is supplied from a gap formed when part of the air-supply tube 62 is released as described above, a closing-preventive member 63 such as a tube-like member or a mesh-like member is inserted in the air-supply tube 62 as shown in FIG. 7. With this, the air-supply tube 62 can be prevented from being completely closed. Also, as shown in FIG. 8, a projection 62a may be provided on an inner wall part of the air-supply tube 62. Furthermore, as shown in FIG. 9, a recessed part 62b may be provided on the inner wall part of the air-supply tube 62. Still further, even if the air-supply tube 62 is pressed onto a V-shaped groove part 130 as shown in FIG. 10, the air-supply tube 62 is not completely closed, and the gas-curtain gas can be supplied from a gap formed at that time. Still further, as shown in FIG. 11, when both ends of a bypass tube 132 are connected to the air-supply tube 62 in advance and the air-supply tube 62 is closed (crushed) by a pressing member 134 (corresponding to the stopper 90 in FIGS. 5A, 5B, and 5C), the gas-curtain gas can also be supplied via the bypass tube 132.

While the endoscope system according to each of the embodiments has been described in detail above, as a matter of course, the present invention is not meant to be restricted to the examples above, and can be variously improved or modified within a range not deviating from the gist of the present invention.

What is claimed is:

1. An endoscope system comprising:
an endoscope insertion part having a window part at an end face;
a fluid tube provided to a tube forming member formed of the endoscope insertion part or a sheath sheathing the endoscope insertion part;
a nozzle provided at a tip of the tube forming member, communicating with the fluid tube, and injecting a fluid supplied from the fluid tube toward the window part of the endoscope insertion part;
a first gas supply tube provided between a gas supply source and the fluid tube and guiding a gas supplied from the gas supply source to the fluid tube;
a second gas supply tube having a limiting device limiting a pressure or a flow rate of the gas supplied from the gas supply source, the second gas supply tube guiding the gas with the pressure or the flow rate limited by the limiting device to the fluid tube; and
a gas switching device provided only to the first gas supply tube between the first and second gas supply tubes and selectively switching between supplying and stopping of the gas to the fluid tube.

2. The endoscope system according to claim 1, wherein
the first gas supply tube is a gas supply tube guiding a gas for cleaning or drying the window part of the endoscope insertion part to the fluid tube, and
the second gas supply tube is a gas supply tube guiding a gas to the fluid tube, the gas for forming a gas curtain on a surface of the window part of the endoscope insertion part and having a pressure or a flow rate lower than the pressure or the flow rate of the gas guided by the first gas supply tube.

3. The endoscope system according to claim 2, further comprising:
a cleaning-fluid supply tube provided between the gas supply source and the fluid tube and guiding a cleaning solution accommodated in an air-tight container to the fluid tube by using the pressure of the gas supplied from the gas supply source; and
a cleaning-fluid switching device provided to the cleaning-fluid supply tube and selectively switching between supplying and stopping of the cleaning solution to the fluid tube.

4. The endoscope system according to claim 3, wherein
the gas switching device and the cleaning-fluid switching device are configured to have timings of opening and closing the gas supply tube and the cleaning-fluid supply tube, respectively, varying according to an amount of operation of an operating member.

5. The endoscope system according to claim 1, wherein
the flow rate of the gas limited by the limiting device and guided by the second gas supply tube is equal to 1/100 or more and equal to 1/50 or less of the gas guided by the first gas supply tube.

6. The endoscope system according to claim 2, wherein
the flow rate of the gas limited by the limiting device and guided by the second gas supply tube is equal to 1/100 or more and equal to 1/50 or less of the gas guided by the first gas supply tube.

7. The endoscope system according to claim 3, wherein
the flow rate of the gas limited by the limiting device and guided by the second gas supply tube is equal to 1/100 or more and equal to 1/50 or less of the gas guided by the first gas supply tube.

8. The endoscope system according to claim 4, wherein
the flow rate of the gas limited by the limiting device and guided by the second gas supply tube is equal to 1/100 or more and equal to 1/50 or less of the gas guided by the first gas supply tube.
